(19) United States
(12) Patent Application Publication
(10) Pub. No.: US 2017/0150950 A1
(43) Pub. Date: Jun. 1, 2017

(54) Medical Instrument

(71) Applicant: Karl Storz GmbH & Co. KG, Tuttlingen (DE)

(72) Inventors: Yann Thouément, Les Essarts le Roi (FR); Régis Besse, Guyancourt (FR); Jochen Stefan, Wald (DE); Daniel Kärcher, Radolfzell (DE)

(21) Appl. No.: 15/360,391

(22) Filed: Nov. 23, 2016

(30) Foreign Application Priority Data
Dec. 1, 2015 (DE) 102015015664.0

(51) Int. Cl. A61B 17/00

(52) U.S. Cl. CPC A61B 17/00; A61B 2017/00367; A61B 2017/00982

(57) ABSTRACT

A medical instrument with a hollow shaft, a handle at the proximal end, and a tool at the distal end such that the tool is rotatable about the longitudinal axis of the shaft via an actuation rod connected to the handle, the actuation rod composed of two parts, with mutually facing end-face toothing arrangements.

### TECHNICAL FIELD

The invention relates to a medical instrument with a hollow shaft, at the proximal end of which a handle is arranged, and at the distal end of which a tool is arranged such that a distal end region of the shaft carrying the tool is designed as a tool tip that can be positioned at an angle with respect to the longitudinal axis of the shaft and such that the tool is rotatable about the longitudinal axis of the shaft respectively about the longitudinal axis of the tool tip, the rotation of the tool about the longitudinal axis of the shaft being effected via an actuation rod which is mounted rotatably in the hollow shaft and which is operatively connected at its proximal end to the handle, the actuation rod being composed of two parts, namely a distal sub-region mounted in the pivotable tool tip and a sub-region mounted in the proximal part of the shaft, and the two mutually facing end faces of the sub-regions of the actuation rod being in engagement with each other at the transition to the pivotable tool tip via end-face toothing arrangements.

### BACKGROUND

Medical instruments for endoscopic surgery generally have a hollow shaft, at the proximal end of which a handle is arranged, and at the distal end of which a tool is arranged. The tool, designed as a gripping, holding and/or cutting instrument, can be actuated via the handle. To be able to provide the greatest possible range of action within the often confined working conditions in which the tool is used, many endoscopic instruments are designed such that the tool can be angled with respect to the longitudinal axis of the shaft and also such that the tool is rotatable about the longitudinal axis of the shaft.

A medical instrument of the type in question is known, for example, from EP 2 036 505 B1. In order to ensure that the end-face toothing arrangements of the sub-regions of the actuation rod can always engage adequately and without jamming, regardless of the degree of the angle of the tooth tip with respect to the shaft of the instrument, the proximal sub-region of the actuation rod in this known medical instrument is pretensioned by a spring element in the direction of the distal sub-region of the actuation rod mounted in the tool tip.

Although this compensation mechanism ensures good engagement of the two end-face toothing arrangements, the structure is very complex and runs counter to the compact configuration that is required particularly in endoscopic instruments. Moreover, the use of spring mechanisms is disadvantageous from the technical point of view of cleaning, since these mechanisms often have undercuts that are difficult to access.

### SUMMARY

Proceeding from this, it is the object of the invention to configure a medical instrument of the aforementioned type which, while having a compact configuration, ensures uniform rotation of jaw parts independently of the angle of the tool tip.

According to the invention the solution to this problem is characterized in that the tooth flanks of the individual teeth of the two end-face toothing arrangements are designed tapering radially outward, and that the tooth flanks of the individual teeth of the two end-face toothing arrangements are additionally designed tapering radially inward.

By designing the tooth flanks of the individual teeth of the two end-face toothing arrangements such that they taper radially outward and in addition radially inward, the teeth on both sides of the end-face toothing arrangements are also prevented from jamming during a pivoting movement of the end-face toothing arrangements relative to each other. This permanent freedom from jamming makes it possible for the first time to do without the axial compensation known from the prior art, for example in the form of spring pretensioning.

By virtue of the additional tapering of the tooth flanks also in the inwardly directed radial direction, the freedom of movement of the individual teeth of the two end-face toothing arrangements relative to each other is further increased.

It is further proposed, in a preferred embodiment of the invention, that the tooth heads of the individual teeth of the two end-face toothing arrangements are rounded. This rounding of the tooth heads, which form the top of the individual teeth, also makes it easier for the end-face toothing arrangements to be pivoted relative to each other without jamming.

According to a further embodiment of the invention, the tooth flanks on both sides of the individual teeth of the two end-face toothing arrangements are composed of flat surface parts and of curved surface parts in order to ensure that, at every angled position of the end-face toothing arrangements relative to each other, force is transmitted from tooth to tooth in the two end-face toothing arrangements in the best possible way and without causing jamming.

Finally, the invention proposes that the radially outer surface parts of the tooth flanks on both sides of the individual teeth of the two end-face toothing arrangements are curved. Designing the radially outer surface parts of the tooth flanks on both sides as curved surface parts allows the toothed wheels to roll on each other without jamming, particularly in the positions in which the end-face toothing arrangements are arranged pivoted relative to each other.

Further features and advantages of the invention will become clear from the attached drawings in which an illustrative embodiment of a medical instrument according to the invention is shown simply by way of example, without limiting the invention to this illustrative embodiment. In the drawings:

### BRIEF DESCRIPTION OF THE DRAWINGS

FIG. 1 shows a perspective side view of a medical instrument according to the invention;

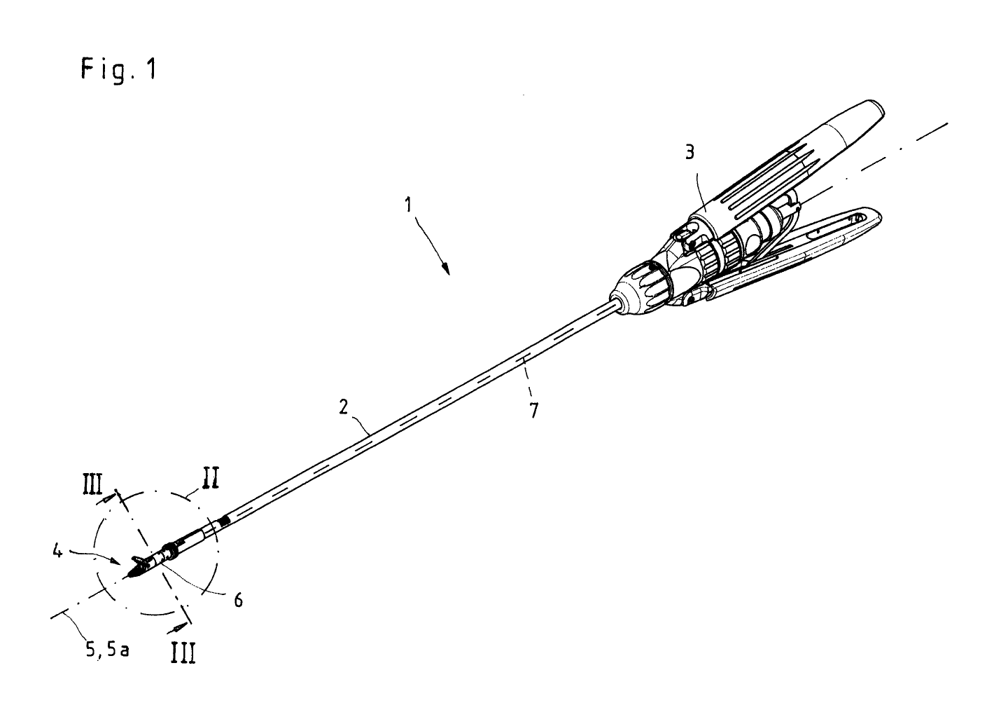

FIG. 2 shows a partially transparent side view of the detail II according to FIG. 1, depicting an angled position;

FIG. 3 shows a sectional detail along the line III-III according to FIG. 1;

FIG. 4 shows a perspective view of an end-face toothing arrangement according to FIG. 2;

FIG. 5 shows a perspective view of the two end-face toothing arrangements according to FIG. 2, in a position in which they are at an angle of 45° to each other, and

FIG. 6 shows a perspective view of the two end-face toothing arrangements according to FIG. 2, in a position in which they are at an angle of 90° to each other.

### DETAILED DESCRIPTION

To give the tool 4 the greatest number of possible degrees of freedom of movement relative to the shaft 2, a distal end region of the shaft 2 carrying the tool 4 is designed as a tool tip 6 that can be positioned at an angle with respect to the longitudinal axis 5 of the shaft 2.

Moreover, the tool 4 is rotatable about the longitudinal axis 5 of the shaft 2, the rotation of the tool 4 about the longitudinal axis 5 of the shaft 2, respectively about the longitudinal axis 5a of the tool tip 6 when the latter is angled, being effected via an actuation rod 7 which is mounted rotatably in the hollow shaft 2 and which is operatively connected at its proximal end to the handle 3, and the actuation rod 7 being composed of two parts, namely a distal sub-region 8 mounted in the pivotable tool tip 6 and a sub-region 9 mounted in the proximal part of the shaft 2.

To form the actuation rod 7, it is possible to use both a solid rod and also a hollow tube.

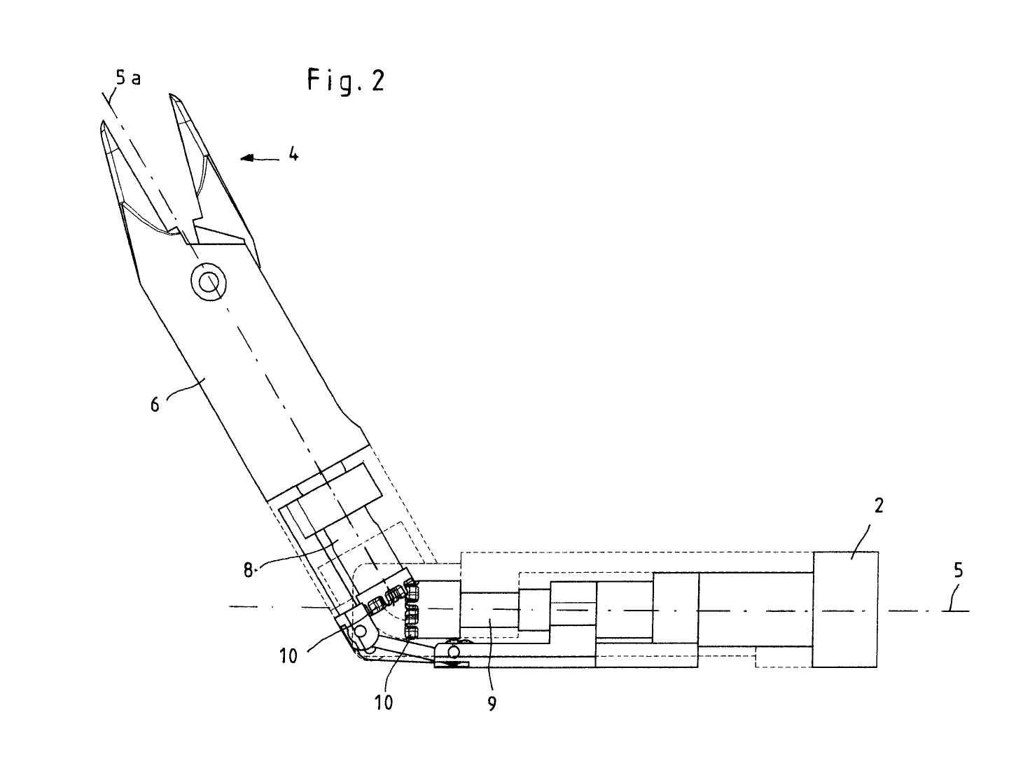

The two mutually facing end faces of the sub-regions 8 and 9 of the actuation rod 7 are in engagement with each other at the transition to the pivotable tool tip 6 via end-face toothing arrangements 10, as can be seen from the schematic cross-sectional view according to FIG. 2. These two end-face toothing arrangements 10 of the two sub-regions 8 and 9 of the actuation rod 7 have the effect that, even in a position in which the two sub-regions 8 and 9 of the actuation rod 7 are arranged at an angle relative to each other, the tool 4 is rotatable about the longitudinal axis 5 of the shaft 2 respectively about the longitudinal axis 5a of the tool tip 6. The end-face toothing arrangements 10 transmit the rotation of the proximal sub-region 9 of the actuation rod 7 about the longitudinal axis 5 of the shaft 2 to the distal sub-region 8 of the actuation rod 7.

FIG. 2 shows the set-up of the two end-face toothing arrangements 10 inside the shaft 2 with the aid of a partially transparent schematic side view in the area of the transition from the proximal shaft 2 to the angled distal tool tip 6.

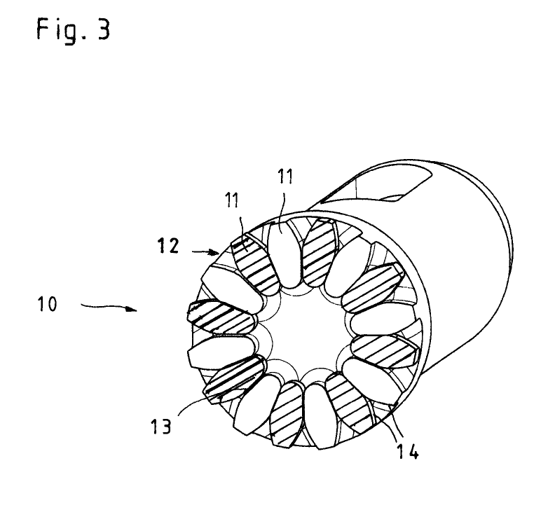

FIG. 3 shows the two end-face toothing arrangements 10 in the non-angled position of the sub-regions 8 and 9 of the actuation rod 7, in which position all the teeth 11 of the respective end-face toothing arrangements 10 of the two sub-regions 8 and 9 of the actuation rod 7 are in engagement with each other.

A main problem in designing the two end-face toothing arrangements 10 of the mutually pivotable sub-regions 8 and 9 of the actuation rod 7 is to ensure that, during the pivoting, the teeth 11 of the end-face toothing arrangements 10 do not jam and block each other.

As can be seen from FIG. 3, the tooth flanks 12 of the individual teeth 11 of the two end-face toothing arrangements 10 are designed tapering radially outward. By designing the tooth flanks 12 of the individual teeth 11 of the two end-face toothing arrangements 10 such that they taper radially outward in relation to the respective toothing arrangement 10, the teeth 11 on both sides of the end-face toothing arrangements 10 are also prevented from jamming during a pivoting movement of the end-face toothing arrangements 10 relative to each other. This permanent freedom from jamming makes it possible to do without axial compensation, for example in the form of spring pretensioning. By virtue of the radially outwardly tapering design of the individual teeth 11 of the two end-face toothing arrangements 10, there is always sufficient lateral clearance for the individual teeth 11, specifically in the radially outer area, with respect to the adjacent teeth 11 of the respective other end-face toothing arrangement 10.

In the end-face toothing arrangements 10, as will be seen from the figures, the tooth flanks 12 of the individual teeth 11 of the two end-face toothing arrangements 10 are additionally designed also to taper radially inward in relation to the respective toothing arrangement 10, as a result of which the freedom of movement of the individual teeth 11 of the two end-face toothing arrangements 10 relative to each other is further increased.

As will further be seen in particular from FIG. 3, the tooth flanks 12 on both sides of the individual teeth 11 of the two end-face toothing arrangements 10 have flat surface parts 13 and curved surface parts 14, wherein the radially outer surface parts 14 of the tooth flanks 12 on both sides of the individual teeth 11 of the two end-face toothing arrangements 10 are curved.

The design of the radially outer surface parts 14 of the tooth flanks 12 as curved surface parts 14 allows the two end-face toothing arrangements 10 to run on each other without jamming, particularly in the positions in which the end-face toothing arrangements 10 are arranged pivoted relative to each other.

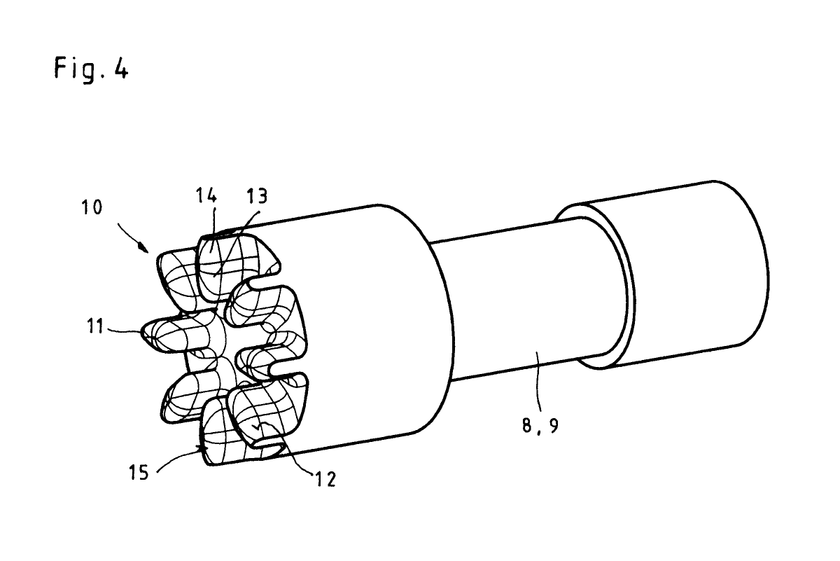

It will also be seen from the perspective view in FIG. 4 that the tooth heads 15 of the individual teeth 11 of the two end-face toothing arrangements 10 are rounded. This rounding of the tooth heads 15, which form the top of the individual teeth 11, also makes it easier for the end-face toothing arrangements 10 to be pivoted relative to each other without jamming.

The end-face toothing arrangements 10 designed in the manner described above are distinguished by the fact that, at all the angled positions of the sub-regions 8 and 9 of the actuation rod 7 relative to each other, the individual teeth 11 are able to run onto each other without jamming, and yet axial compensation can be omitted.

On account of the different angle settings of the end-face toothing arrangements 10 relative to each other, an involute toothing has to be dispensed with. By virtue of the design of the radially outwardly tapering tooth flanks 12 of the individual teeth 11 of the two end-face toothing arrangements 10 and the configuration of the tooth flanks 12 on both sides of the individual teeth 11 from flat surface parts 13 and curved surface parts 14, the geometry of the end-face toothing arrangements 10 is such that, at all the angled positions to which they are pivoted relative to each other, the curved surface parts 14 of one end-face toothing arrangement 10 run, in a manner free from jamming, either on the flat surface parts 13 or the curved surface parts 14 of the other end-face toothing arrangement 10.

In the non-pivoted orientation of the sub-regions 8 and 9 of the actuation rod 7 relative to each other, as shown in FIG. 3, the teeth 11 engage in one another without the need for rolling of the tooth flanks 12. In this position, the flat surface parts 13 of the tooth flanks 12 bear on each other.

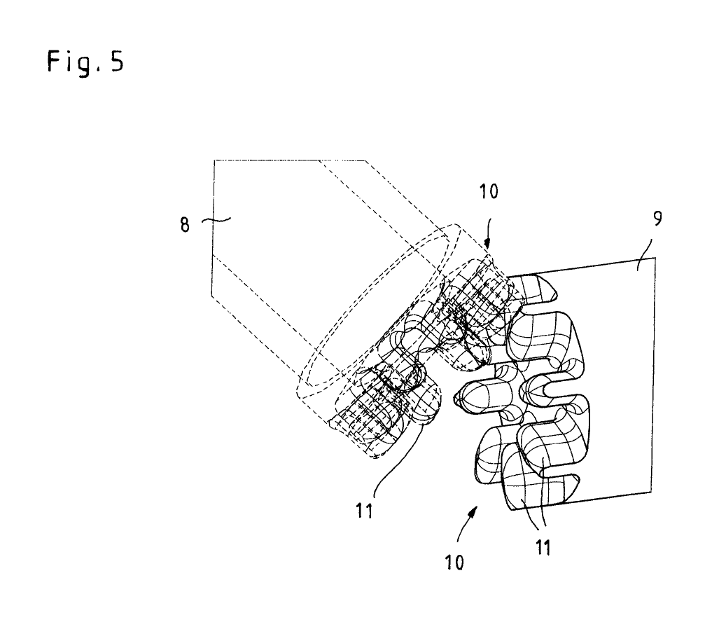

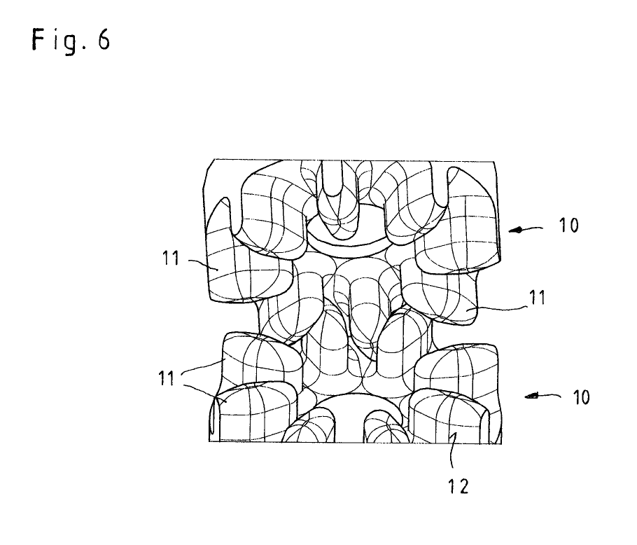

FIGS. 5 and 6 show the end-face toothing arrangements 10 in a position in which said end-face toothing arrangements 10, respectively the sub-regions 8 and 9 of the actuation rod 7, are pivoted relative to each other by 45° (FIG. 5) and by 90° (FIG. 6). In these positions in which they are pivoted relative to each other, the teeth 11 of the end-face toothing arrangements 10 roll on each other only on the curved surface parts 14 of the tooth flanks 12 and thus transmit the torsional moment from the proximal sub-region 9 of the actuation rod 7 to the distal sub-region 8 of the actuation rod 7, with at least one tooth 11 of one end-face toothing arrangement 10 always being in engagement with a tooth 11 of the other end-face toothing arrangement 10.

1. A medical instrument with a hollow shaft, at the proximal end of which a handle is arranged, and at the distal end of which a tool is arranged such that a distal end region of the shaft carrying the tool is designed as a tool tip that can be positioned at an angle with respect to the longitudinal axis of the shaft and such that the tool is rotatable about the longitudinal axis of the shaft respectively about the longitudinal axis of the tool tip, the rotation of the tool about the longitudinal axis of the shaft being effected via an actuation rod which is mounted rotatably in the hollow shaft and which is operatively connected at its proximal end to the handle, the actuation rod being composed of two parts, namely a distal sub-region mounted in the pivotable tool tip and a sub-region mounted in the proximal part of the shaft, and the two mutually facing end faces of the sub-regions of the actuation rod being in engagement with each other at the transition to the pivotable tool tip via end-face toothing arrangements,
wherein the tooth flanks of the individual teeth of the two end-face toothing arrangements are designed tapering radially outward, and the tooth flanks of the individual teeth of the two end-face toothing arrangements are additionally designed tapering radially inward.

2. The medical instrument as claimed in claim 1, wherein the tooth heads of the individual teeth of the two end-face toothing arrangements are rounded.

3. The medical instrument as claimed in claim 1, wherein the tooth flanks on both sides of the individual teeth of the two end-face toothing arrangements are composed of flat surface parts and of curved surface parts.

4. The medical instrument as claimed in claim 3, wherein the radially outward surface parts of the tooth flanks on both sides of the individual teeth of the two end-face toothing arrangements are curved.

5. The medical instrument as claimed in claim 2, wherein the tooth flanks on both sides of the individual teeth of the two end-face toothing arrangements are composed of flat surface parts and of curved surface parts.
